(19) United States
(12) Patent Application Publication
(10) Pub. No.: US 2026/0174214 A1
(43) Pub. Date: Jun. 25, 2026

(54) Cosmetic Eye Mask

(71) Applicant: YA-MAN LTD., Tokyo (JP)

(72) Inventors: Masayuki KANEKO, Tokyo (JP); Masao SEIMIYA, Tokyo (JP)

(21) Appl. No.: 18/987,036

(22) Filed: Dec. 19, 2024

(51) Int. Cl. A45D 44/22; A61N 1/04; A61N 1/32

(52) U.S. Cl. CPC A45D 44/22; A61N 1/0452; A61N 1/0484; A61N 1/328

(57) ABSTRACT

A cosmetic eye mask used by being brought into contact with an area around eyes, includes a mask body including accommodations, the accommodations being disposed at positions corresponding to positions around respective eyes, and stimulators accommodated in the respective accommodations. Each stimulator includes a first electrode disposed at a position corresponding to a position on a lateral side of a corresponding eye and at an outer part of a face, and a second electrode disposed at a position corresponding to a position on a lower side of the corresponding eye in such a manner that a longitudinal direction of the second electrode elongates along the corresponding eye. Each stimulator is configured to apply electrical stimulation to a human body by passing electricity between the first electrode and the second electrode. The second electrode includes a conductive second electrode body and a conductive thin sheet accommodated along a longitudinal direction of the second electrode body.

### BACKGROUND

### TECHNICAL FIELD

The present disclosure relates to a cosmetic eye mask.

### RELATED ART

There have been cosmetic eye masks (cosmetic masks for around eyes) configured to apply electrical stimulation to an area around eyes (e.g., JP 2004-216069 A).

However, conventional cosmetic eye masks have room for improvement in terms of fitness to the face, electrode shapes, etc. In particular, it is hard to say that such cosmetic eye masks can apply sufficient electrical stimulation to the muscles from the outer corner of the eye to the inner corner side of the eye, where signs of aging tend to be more noticeable.

In view of the above circumstances, the present disclosure provides a cosmetic eye mask (cosmetic mask for area around eyes) capable of applying sufficient electrical stimulation along from the outer corner of eye to the inner corner side of eye.

### SUMMARY

An aspect of the present disclosure provides a cosmetic eye mask used in a manner such that the cosmetic eye mask is brought into contact with an area around eyes. The cosmetic eye mask includes a mask body including accommodations, the accommodations being disposed at positions corresponding to positions around respective eyes, and stimulators accommodated in the respective accommodations. Each stimulator includes a first electrode disposed at a position corresponding to a position on a lateral side of a corresponding eye and at an outer part of a face, and a second electrode disposed at a position corresponding to a position on a lower side of the corresponding eye in such a manner that a longitudinal direction of the second electrode elongates along the corresponding eye. Each stimulator is configured to apply electrical stimulation to a human body by passing electricity between the first electrode and the second electrode. The second electrode includes a conductive second electrode body and a conductive thin sheet accommodated along a longitudinal direction of the second electrode body.

Such an aspect can provide a cosmetic eye mask capable of applying electrical stimulation well from the outer corner of the eye to the inner corner side of the eye.

### BRIEF DESCRIPTION OF THE DRAWINGS

FIG. 1 is a perspective view of a cosmetic eye mask viewed from the side opposite to a skin surface of a user.

FIG. 2 is a view of the cosmetic eye mask from a skin surface side of the user.

FIG. 3 is a schematic diagram of a stimulator viewed from the skin surface side.

FIG. 4 is a sectional view at a line A-A’ of FIG. 3.

FIG. 5 is a sectional view at a line B-B’ of FIG. 3.

### DETAILED DESCRIPTION

With reference to the drawings, a detailed description will be given of the cosmetic eye mask 1 according to the present disclosure.

In this specification, the upper side and the lower side of the cosmetic eye mask 1 are defined as represented in each drawing. The center side (the side in the direction of the inner corner of eye) of the cosmetic eye mask 1 is referred to as “inner side” and an outer side (the side in the direction of the outer corner of eye) of the cosmetic eye mask 1 is also referred to as “outer side＂.

### COSMETIC EYE MASK

### Overall Configuration

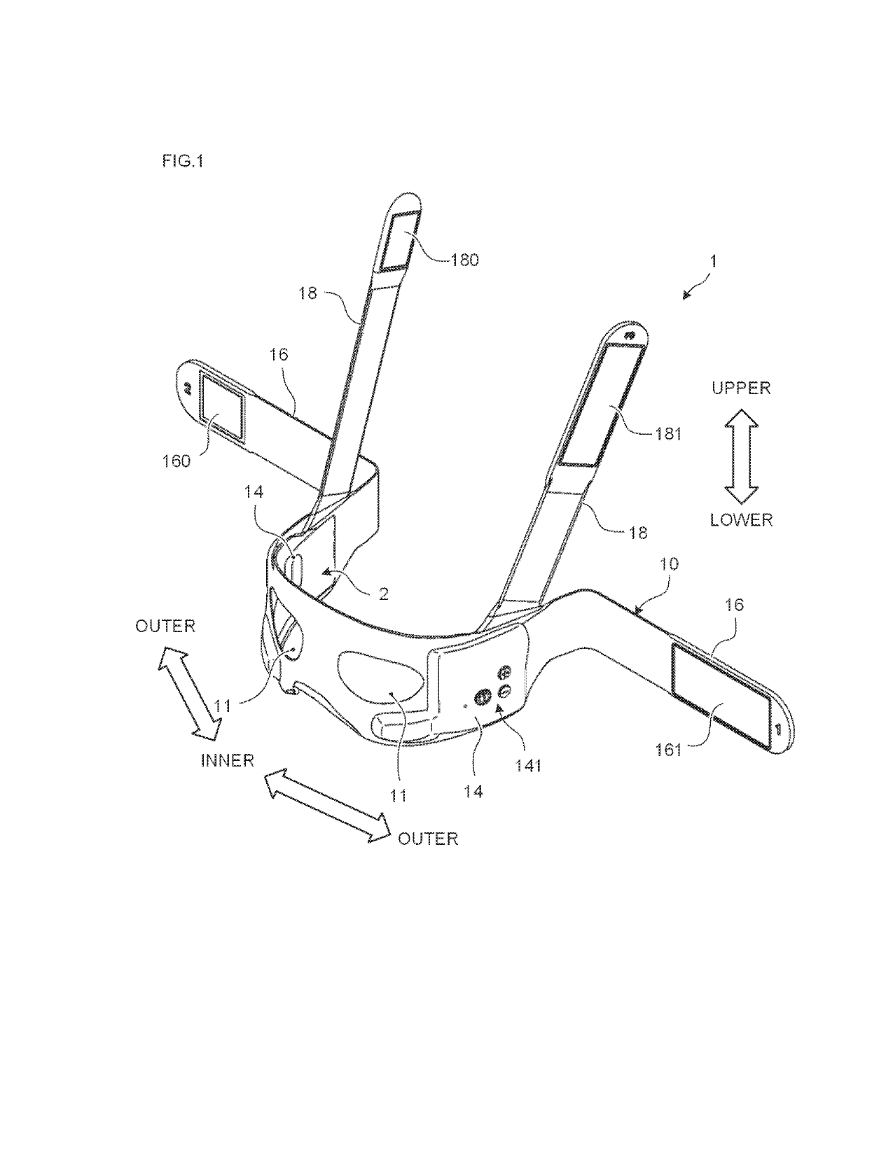

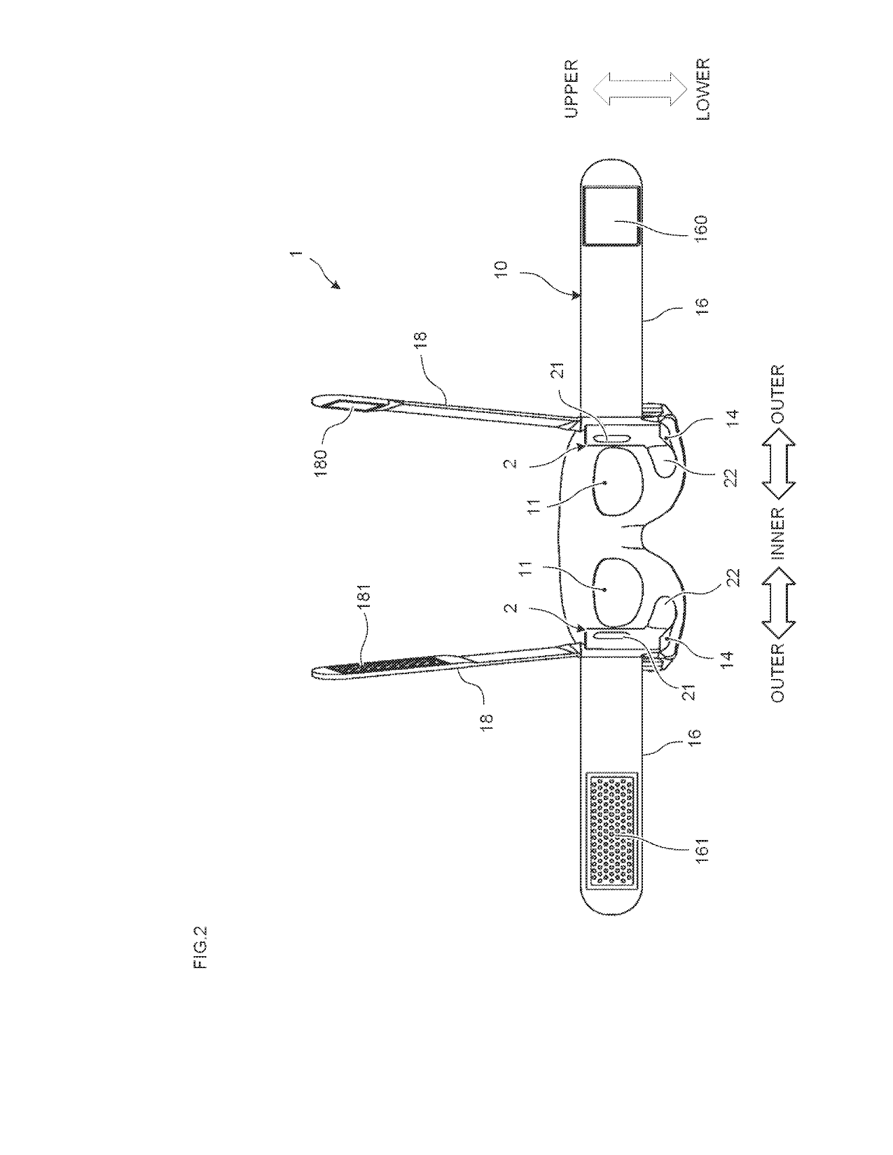

First, with reference to FIG. 1 to FIG. 2, a description will be given of the overall configuration of the cosmetic eye mask 1. FIG. 1 is a perspective view of a cosmetic eye mask viewed from the side opposite to a skin surface of a user. FIG. 2 is a view of the cosmetic eye mask from a skin surface side of the user.

When used, the cosmetic eye mask 1 of the present disclosure is a cosmetic mask brought into contact with skin surface of a user, especially skin surface around eyes. As illustrated in FIG. 1, etc., the cosmetic eye mask 1 includes a mask body 10 and a pair of stimulators 2.

The mask body 10 is used by wrapping it (i.e., being attached to) around the circumference of the user’s head, including the area around the eyes of the user. The mask body 10 includes (or is made of) an elastic material (or a stretchable material), such as silicone rubber. The mask body 10 includes a pair of accommodations 14, which are shaped as indentations (or depressions) disposed, when the mask is worn, at positions corresponding to the vicinity of respective eyes and facing the user’s skin surface.

A pair of stimulators 2 is used in a state where the stimulators 2 are detachably accommodated in the respective accommodations 14, i.e., where the stimulators 2 are placed between the mask body 10 and the user. When used, the pair of stimulators 2 can apply electrical stimulation to (i.e., electrically stimulate) the user’s skin surface and its inner muscles and/or fascia. When electrical stimulation is applied to muscles and/or fascia, the muscles and/or fascia are encouraged to contract and/or relax, which promotes blood and lymph circulation (or flow) therearound. This can be expected to exhibit effects such as relieving muscle stiffness, strengthening muscles, discharging waste products, and the like.

As described above, the cosmetic eye mask 1 is used while the stimulators 2 for applying electrical stimulation are detachably attached to the mask body 10 having elasticity. Thereby, the stimulators 2 can be brought into close contact with the user’s head, especially in the vicinity of the eyes, regardless of the shape of the user’s head.

Since the stimulators 2 are detachably attachable to the mask body 10, even when the mask body 10 deteriorates, only the mask body 10 needs to be replaced and there is no need to replace the stimulators 2. In other words, it is possible to reduce the environmental load caused by continued use of the cosmetic eye mask 1.

### Mask Body

Next, a description will be given of the mask body 10. As illustrated in FIG. 1 and FIG. 2, the mask body 10 includes a pair of holes 11 on the inner side of the pair of accommodations 14. The holes 11 are disposed at positions corresponding to the user’s eyes when the mask body 10 is worn (or attached). This allows the user to use the cosmetic eye mask 1 while working on other tasks, facilitating making the use of the cosmetic eye mask 1 a habit.

The mask body 10 includes a pair of lateral (left-right) attachment bodies 16 extending toward the outer side than the accommodations 14. The mask body 10 can be attached (or secured or fixed) to the user’s head by wrapping the user’s head, especially the rear part of the head, with the lateral attachment bodies 16. Each lateral attachment body 16 has a fastener 160 or a fastener 161 at its end, and when these are stuck (or joined or attached) to each other, the wrapping lateral attachment body 16 is positioned relative to the user’s head.

The mask body 10 further includes a pair of upper attachment bodies 18 extending toward the upper side than the accommodations 14. The mask body 10 can be attached (or secured or fixed) to the user’s head by wrapping the user’s head, especially the upper part of the head, with the upper attachment bodies 18. Each upper attachment body 18 has a fastener 180 or a fastener 181 at its end, and when these are joined (or stuck or attached) to each other, the wrapping upper attachment body 18 is positioned relative to the user’s head.

In this manner, since the user’s head is wrapped with the lateral attachment bodies 16 and the upper attachment bodies 18 at different positions and at different angles, the mask body 10 can be attached (fixed) on the user’s head without shift, displacement, or slip. This further facilitates making a habit of using the cosmetic eye mask 1, since such problems hardly happen that the mask body 10 shifts and makes the user feel uncomfortable. In addition, this improves the reliability of fitness (close contact) between the user’s skin surface and the stimulator 2 attached to the mask body 10.

The detailed configurations of the fasteners 160 and 161 on the lateral attachment body 16 and the fasteners 180 and 181 on the upper attachment body 18 are not particularly limited as long as they can be attached to each other. For example, they may be hook-and-loop fasteners (i.e., so-called Velcros), snap buttons, or the like.

The accommodations 14 of the mask body 10 are disposed at positions corresponding to the vicinity of respective eyes, as described above. More specifically, the accommodations 14 are disposed on the outer side of the holes 11, which are disposed at positions corresponding to respective eyes, that is, the accommodations 14 are disposed near the left and right outer corners of the eyes. Each of the accommodations 14 has a shape of an indentation (or a depression) facing the skin surface, and the stimulator 2 can be accommodated (fit) in this indentation. Each of the accommodations 14 protrudes on the side opposite to the skin surface, and has a convex operation unit 141, such as buttons, on the protruding area. By pressing the operation unit 141, the user can operate the stimulator 2, which is detachably attached on its the skin surface side.

### Stimulator

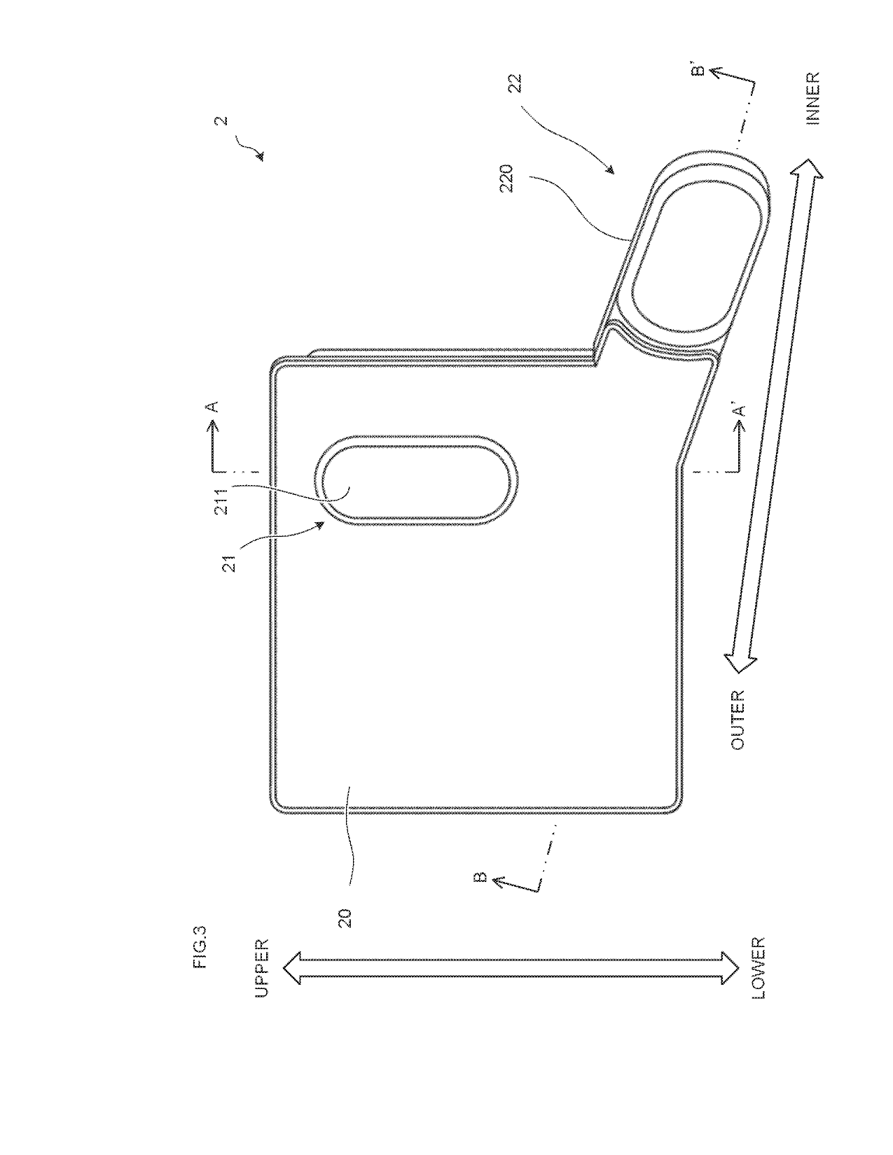

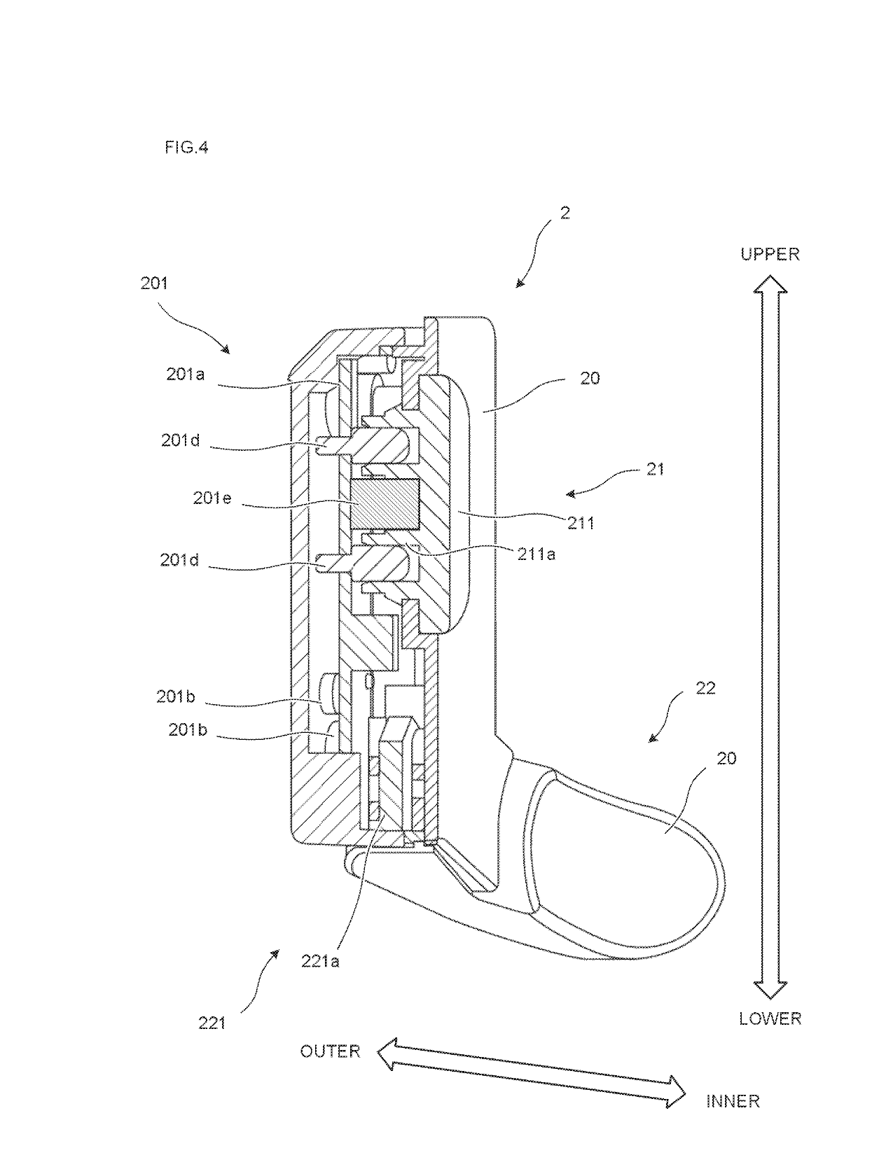

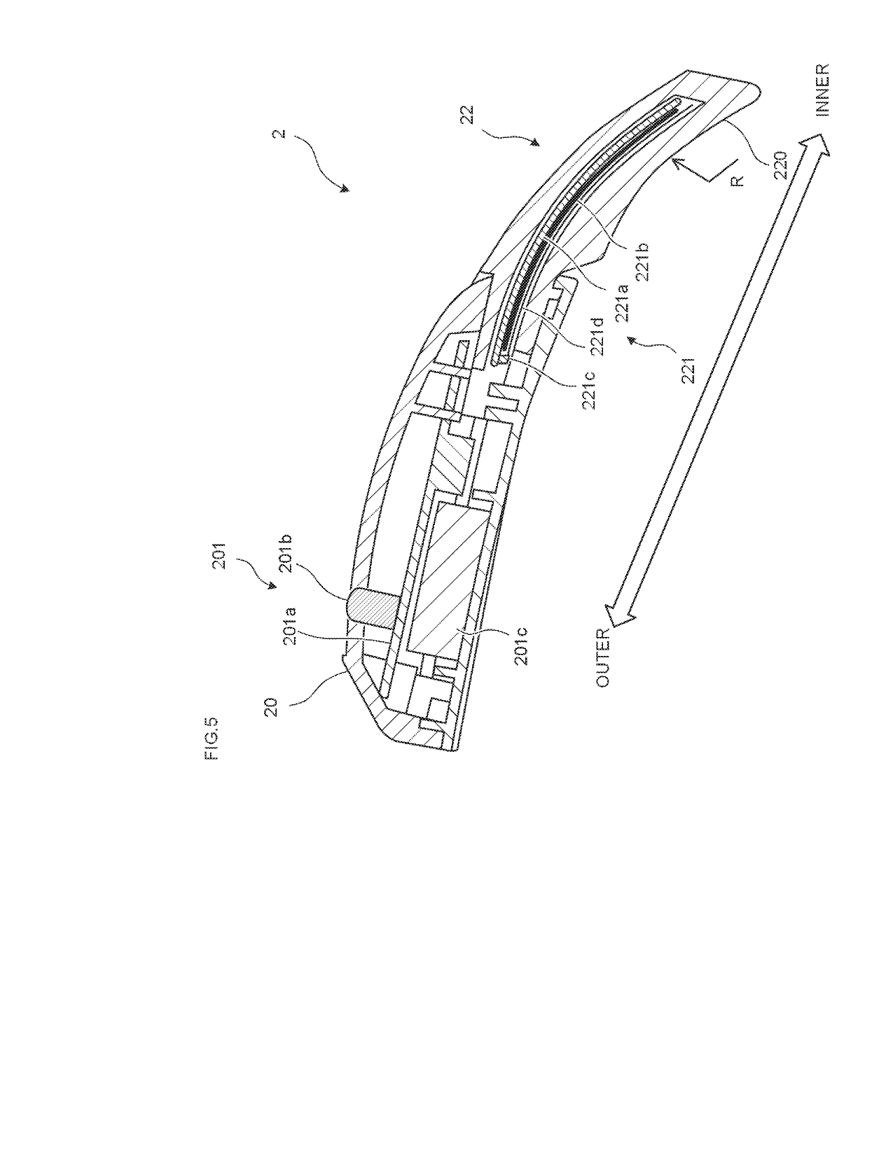

Next, a description will be given of the stimulator 2 with reference to FIG. 3 to FIG. 5. FIG. 3 is a schematic diagram of a stimulator viewed from the skin surface side. FIG. 4 is a sectional view at a line A-A’ of FIG. 3. FIG. 5 is a sectional view at a line B-B’ of FIG. 3. When used, the pair of stimulators 2 is accommodated in the accommodations 14 disposed at positions respectively corresponding to the vicinity of the left and right eye corners. Hereinafter, a description will be given of the stimulator 2 accommodated on the left eye side (front side of the drawing in FIG. 1), but the stimulator 2 accommodated on the right eye side (deep side of the drawing in FIG. 1) has the same or similar configurations and functions.

### External Configuration

As illustrated in FIG. 3, the stimulator 2 includes an electrode support 20. The electrode support 20 is made of (or includes) a hard resin material such as polycarbonate, polypropylene, ABS resin, or any combination thereof, for example. This enables protection of various electronic components housed into the electrode support 20 while avoiding the electrode support 20 from being heavy. The electrode support 20 has a small electrical conductivity (or a property hardly or never conducting electricity). This prevents electrical discharges from unintended points, enabling electrical stimulation to be concentrated and applied to the skin surface from each electrode (described below).

The stimulator 2 includes a first electrode 21 and a second electrode 22, which are supported by the electrode support 20. A first electrode body 211 of the first electrode 21 is exposed from the electrode support 20 at a position corresponding to the vicinity of the outer corner of an eye (a position on the lateral side of the corresponding (left) eye and at the outer part of the face). The second electrode body 220 of the second electrode 22 extends from the electrode support 20 toward the inner side (i.e., in the direction of the inner corner of the eye). In more detail, the longitudinal direction of the second electrode body 220 extends along each eye at a position corresponding to a position on the lower side of (i.e., under) each eye.

The first electrode body 211 and the second electrode body 220 are conductive and are configured to transmit/receive (conduct) electric power applied from a power supply unit 201c (described below) in the stimulator 2 to each other via the user’s skin surface and its inner muscles or fascia. In other words, the stimulator 2 is configured to be capable of applying electrical stimulation to the user (human body) by passing the electricity between the first electrode 21 and the second electrode 22.

Thus, by passing (conducting) electricity between the first electrode 21 disposed near the outer corner of an eye and the second electrode 22 disposed under the eye, electrical stimulation can be applied especially to the orbicularis oculi muscle and the temporalis muscle (and/or other cutaneous muscles located around the temporal region).

The first electrode body 211 and/or the second electrode body 220 may be made of (or include) an elastic material that is comfortable to and gently touches the skin. This allows them to make contact with the user’s skin surface with gentle forces, thus preventing damage to the skin surface. In particular, at least the second electrode body 220 may be mainly composed of conductive medical-grade silicone. This can more effectively prevent damage to the particularly delicate skin surface under the eye.

Furthermore, both the first electrode body 211 and the second electrode body 220 may be mainly composed of conductive medical-grade silicone. In this case, even when the cosmetic eye mask 1 is used after applying a cosmetic product such as a beauty serum, etc., the physical properties of the first electrode body 211 and the second electrode body 220 are not likely to change (or be deteriorated), encouraging long-period use of the cosmetic eye mask 1 (the first electrode body 211 and the second electrode body 220). In addition, since the surfaces of the first electrode body 211 and the second electrode body 220 are not likely to deteriorate and become uneven, they can be used hygienically by simply wiping off cosmetic products.

The conductivity of medical grade silicone may be due to the structure of the silicone molecules themselves (e.g., including introduced conductive substituents, etc.), or may be realized by conductive fillers dispersed between silicone molecules.

The first electrode body 211 of the first electrode 21 extends along the vertical direction (eye height direction). This enables electrical stimulation along the inner-side (eye-side) edge of the temporal fossa, where the temporalis muscle is located. The first electrode body 211 forms a surface slightly protruding from the stimulator 2 to the user’s skin surface. The skin surface around the temples may be slightly concave (or sunken or hollow) inside the heads of some users because of weakening of the temporalis muscle, etc. Even in a case where the user’s skin surface is concave, the protrusion of the first electrode body 211 facilitates its contact with the user’s skin surface and application of electrical stimulation to the temporalis muscle, etc.

The second electrode body 220 of the second electrode 22 extends out from the electrode support 20 as described above. Therefore, it is possible to deform the second electrode body 220 along the shape of the area under the eye, where individual differences are particularly large, facilitating close contact of the second electrode body 220 with the skin surface. In other words, electrical stimulation can be effectively applied to the orbicularis oculi muscle under the eyes, which is likely to be affected by aging. As illustrated in FIG. 5, etc., the second electrode body 220 curves toward the skin surface along its longitudinal direction. Thus, adjusting the degree of curvature (curvature R in FIG. 5) of the curved surface of the second electrode body 220 can allow the second electrode body 220 to closely contact with the skin surface with comfortable fitness to the shape of the cheekbone.

The Shore A hardness of the second electrode body 220 may be in a range from 30 Hs to 60 Hs, or from 40 Hs to 50 Hs. Such hardness can make contact feeling moderate when the second electrode body 220 contacts to the skin surface. Since the second electrode body 220 is not too hard, it can easily contact closely to the shape under the user’s eye regardless of individual differences thereof. In addition, since the second electrode body 220 is not too soft, its elastic force moderately acts when it contacts the user’s skin surface. Therefore, the curved surface of the second electrode body 220 can be deformed along and fitted well to the roundness under the user’s eyes. In addition, components accommodated in the second electrode body 220 can be protected well and prevented from unintentionally displaced.

The Shore A hardness can be obtained using a durometer type A (spring type A) in accordance with ISO 7619-2003 (or the corresponding JIS 6253-3:2012).

### Internal Configuration

As illustrated in FIG. 4 and FIG. 5, etc., the stimulator 2 includes a controller 201 inside it. The controller 201 is configured to control the operation of the first electrode 21 and the second electrode 22. The controller 201 includes a first substrate 201a and a reception unit 201b (operation unit), a power supply unit 201c, an application unit 201d, and a warm-up unit 201e, which are mounted on the first substrate201a.

The first substrate 201a is not particularly limited, but may be, for example, a rigid substrate such as a glass epoxy substrate. In this case, it is possible to mount various electronic components onto the first substrate 201a and thereby to provide a highly functional cosmetic eye mask 1. The power supply unit 201c is, for example, a rechargeable battery (secondary battery) such as a lithium-ion battery, and is configured to supply power to the entire stimulator 2. The application unit 201d is an electric contact configured to apply electric power to the first electrode body 211. The warm-up unit 201e is configured to provide heat to the first electrode body 211.

The reception unit 201b is configured to receive an operation by the user so that the controller 201 can provide a function based on the operation. Specifically, the reception unit 201b is disposed at a position corresponding to the operation unit 141 of the mask body 10 illustrated in FIG. 1, and the configuration is such that, when the user presses the operation unit 141, the reception unit 201b is also pressed. When the reception unit 201b is pressed, the controller 201 provides changes in on/off of power of the stimulator 2, the intensity of the electrical stimulation, the mode of the electrical stimulation, etc. The numbers of the operation unit 141 and the reception unit 201b are not limited to those illustrated in the drawings, as long as the numbers are such that the user can intuitively operate them. Furthermore, the mechanism of how the reception unit 201b receives operations is not limited to this, as long as the output of the controller 201 can be changed based on the operations by the user.

The controller 201 is configured to switch a mode to (or between) the first mode and the second mode in response to an operation by the user on the operation unit 141 and the reception unit 201b (operation unit). The first mode and the second mode differ in the muscles around the eyes to which greater electrical stimulation is applied. As described above, the cosmetic eye mask 1 can switch the mode to the first mode and the second mode, thus more appropriately responding to the user’s needs. The part of the body to be especially cared for may differ depending on the user, or even the same user may want to focus on different parts depending on the conditions of the day.

Specifically, by switching the electrode to which voltage is first applied (i.e., from which voltage application started) between the first electrode 21 and the second electrode 22, the controller 201 switches the mode between the first mode and the second mode. Thus, since the controller 201 can switch the mode by such a simple control, there is no need to install complicated electronic circuits, and the stimulator 2 itself can be prevented from becoming larger.

The inventors of the present disclosure have found that, as a result of diligent investigation, electrical stimulation (strong electric current) of higher intensity is applied to the muscles nearer to the electrode to which the voltage is first applied, and that the nearer muscles can be more effectively loosen or relaxed. The same applies regardless of whether the current is direct current or alternating current.

More specifically, in the first mode, the controller 201 is configured to apply voltage first to (i.e., start applying voltage from) the first electrode 21 and thereby apply greater electrical stimulation to the temporalis muscle. In the second mode, the controller 201 is configured to apply voltage first to (i.e., start applying voltage from) the second electrode 22 and thereby apply greater electrical stimulation to the orbicularis oculi muscle.

Thus, by a simple control, the cosmetic eye mask 1 can change (switch) the muscle to which greater electrical stimulation is applied between the temporalis muscle and the orbicularis oculi muscle. For example, in a case where the user wants to improve headaches, teeth clenching, etc. in addition to dullness around the eyes, the user can select the first mode to focus on the temporalis muscle care. On the other hand, in a case where the user wants to focus on improving dark circles, wrinkles, eye strain, etc., the user can select the second mode to focus on the orbicularis oculi muscle care.

As illustrated in FIG. 4, the application unit 201d configured to apply a voltage to the first electrode body 211 is in contact with the first electrode body 211 inside the stimulator 2. The number of the application units 201d may be two as illustrated in FIG. 4, or may be one or three or more. Regardless of the number of the application units 201d, the application unit 201d may be disposed along the longitudinal direction of the first electrode body 211. This enables more uniform electrical stimulation to be applied to the skin surface along the longitudinal direction of the first electrode body 211. For example, when there is one application unit 201d, the application unit 201d may extend along the longitudinal direction of the first electrode body 211.

The first electrode body 211 further has a wall 211a for each application unit 201d. The wall 211a protrudes from the first electrode body 211 toward the interior of the electrode support 20, and is configured to contact the application unit 201d while surrounding its periphery. This increases the contact area between the application unit 201d and the first electrode body 211, facilitating current supply to the first electrode body 211. In addition, since the electric current supplied from the application unit 201d once passes through the wall 211a before reaching the outermost surface of the first electrode body 211, hindering the electric current from concentrating too much at a specific point on the outermost surface of the first electrode body 211. This can prevent too strong electrical stimulation from being applied to a single point on the skin surface. In other words, the electric current can easily diffuse more uniformly on the outermost surface of the first electrode body 211, and more uniform electrical stimulation can be applied to the skin surface in contact with the outermost surface.

The warm-up unit 201e is also in contact with the first electrode body 211 inside the stimulator 2. The warm-up unit 201e is not particularly limited, but may be, for example, a ceramic heater or a film heater. The warm-up unit 201e can warm the skin surface and its interior via the first electrode body 211, realizing loosening or relaxing of the temporalis muscle and the like by both electrical stimulation and thermal stimulation (warmth). The warm-up unit 201e may also extend along the longitudinal direction of the first electrode body 211 as well as the application unit 201d.

A controller 221 is accommodated inside the second electrode body 220 of the second electrode 22, as illustrated in FIG. 5. The controller 221 includes a second substrate 221a, a warm-up unit 221b and an application unit 221c, which are mounted on the second substrate 221a, and a conductive thin sheet 221d in contact (or connected) with the application unit 221c. The controller 221 is electrically connected to the controller 201.

The second substrate 221a is a flexible substrate. This allows the second substrate 221a to deform with the second electrode body 220 bending along the shape under the eye. In addition, since the second substrate 221a has a certain degree of rigidity, the second substrate 221a can exert a moderate repulsive force when the second electrode body 220, which curved to the skin surface, is placed along the cheek bone.

The warm-up unit 221b mounted on the second substrate 221a is a film heater, and is disposed between the second substrate 221a and the second electrode body 220 along the longitudinal direction of the second electrode body 220. Such disposition of the warm-up unit 221b can relax or loosen the orbicularis oculi muscle, which is likely to influence the impression of the eye, along its lower edge. In addition, using a film heater as the warm-up unit 221b can allow itself to be flexibly deformed together with the second electrode body 220, further facilitating close contact of the second electrode body 220 and the skin surface.

The application unit 221c is an electric contact configured to apply electric power to the second electrode body 220. The application unit 221c contacts to or is connected to at least a thin sheet 221d, and is configured to apply electric power to the second electrode body 220 via this thin sheet 221d.

The conductive thin sheet 221d is accommodated along the longitudinal direction of the second electrode body 220. As a result, the electric current supplied from the application unit 221c can diffuse along the longitudinal direction of the second electrode body 220 via the thin sheet 221d. In other words, the current can flow relatively uniformly to the tip end of the second electrode body 220. In other words, sufficient electrical stimulation can be applied from the outer corner of the eye to the inner corner side of the eye.

It also can be said that the thin sheet 221d is disposed between the warm-up unit 221b and the second electrode body 220. Therefore, the heat of warm-up unit 221b can also be transferred to the tip end of the second electrode body 220.

In other words, the thin sheet 221d can allow electrical stimulation and thermal stimulation (warmth) to be diffused from the outer corner of the eye to the inner corner side of the eye without requiring a complicated configuration.

The thin sheet 221d is a thin component that bends flexibly, such as a membrane, layer, sheet, woven fabric, foil, etc. Therefore, the thin sheet 221d does not prevent the deformation of the second electrode body 220. Thus, the second electrode body 220 can be brought into close contact with the skin surface well.

The thin sheet 221d may especially be a metal mesh. In this case, the thin sheet 221d can have moderate rigidity while being flexible. Therefore, even when the second electrode body 220 is repeatedly bent during use, it can be expected that the thin sheet 221d will continue to be located along the longitudinal direction of the second electrode body 220 without folding (or wrinkling, creasing, or rumpling). Even after repeated use of the cosmetic eye mask 1, it is possible to continue to apply electrical stimulation even to the orbicularis oculi muscle near the inner corner of the eye.

The material and detailed composition of the thin sheet 221d are not particularly limited, but it may be obtained, for example, by weaving fine threads made of copper, aluminum, silver, gold, or alloys of these materials in a loose pattern in both warp and weft directions.

However, the flexural rigidity of the thin sheet 221d is smaller than the flexural rigidity of the second electrode body 220 (the flexural rigidity of the second electrode body 220 is greater than the flexural rigidity of the thin sheet). Flexural rigidity is an expression of the resistance to deformation when a force is applied, and can be measured by the amount of deflection caused by an external force. In other words, the thin sheet 221d bends with less force than the second electrode body 220. Hence, when the second electrode body 220 contacts the skin surface, the thin sheet 221d can follow the deformation of the second electrode body 220 without hindering the deformation.

Such a cosmetic eye mask 1 can apply electrical stimulation to the user’s face skin surface and its inner muscles or fascia, with the first electrode body 211 and the second electrode body 220 placed in close contact with the skin surface. In particular, the above-described configuration of the second electrode body 220 can allow the second electrode body 220 to closely contact well with the skin surface from the outer corner of the eye to the inner corner side of the eye, enabling application of electrical stimulation to the tip side of the second electrode 22 (the inner corner side of the eye).

This can promote blood and lymph circulation (or flow) by encouraging contraction and/or relaxation of the muscles around the eyes. This can be expected to further improve the skin firmness of the eye area, improve dark circles, reduce eye strain, eliminate squareness of jaw caused by teeth clenching, and the like.

### MODIFICATION EXAMPLES

The following describes modification examples of the above-described cosmetic eye mask 1. Each of the descriptions of the above embodiment and the following modification examples can be combined with each other.

The shape of the mask body 10 is not limited to the example of the embodiment described above, as long as the stimulator 2 can be fixed near the eyes in a hands-free manner. For example, the mask body 10 may not have the holes 11 at a position corresponding to respective eyes. In this case, the user is prevented from using his/her eyes while using the mask body 10, thereby preventing further accumulation of eye strain. For example, the mask body 10 may not have the lateral attachment bodies 16 or the upper attachment bodies 18.

In the above-described embodiment, a description is given of an example of configuring the stimulator 2, the first electrode 21 and the second electrode 22 as a whole, but the present disclosure is not limited to this. For example, the first electrode 21 and the second electrode 22 may be separately formed, and a stimulator 2 may be provided with each of them.

The controller 201 is not limited to only having the first mode and the second mode described in the embodiment, and the controller 201 may further have another mode.

Furthermore, in the above-described embodiment, a description is given of an example where the controller 201 and the controller 221 have the warm-up unit 201e and the warm-up unit 221b, respectively, but the present disclosure is not limited to this. The warm-up unit 201e and the warm-up unit 221b may be omitted, as long as the cosmetic eye mask 1 can apply electrical stimulation. The other components may also be omitted, replaced, or added, as well as the warm-up unit 201e and the warm-up unit 221b.

In the above-described embodiment, a description is given of an example where the thin sheet 221d connected to the application unit 221c is accommodated in the second electrode body 220, but the present disclosure is not limited to this. The thin sheet 221d may be omitted. In this case, the application unit 221c may be formed as a conductive pattern exposed on a surface of the second substrate 221a on the skin surface. Then, an electric current is supplied directly from this conductive pattern (application unit 221c) to the second electrode body 220.

The present disclosure may be provided in each of the following aspects.

(1) A cosmetic eye mask used in a manner such that the cosmetic eye mask is brought into contact with an area around eyes, the cosmetic eye mask comprising: a mask body including accommodations, the accommodations being disposed at positions corresponding to positions around respective eyes; and stimulators accommodated in the respective accommodations, each stimulator including: a first electrode disposed at a position corresponding to a position on a lateral side of a corresponding eye and at an outer part of a face; and a second electrode disposed at a position corresponding to a position on a lower side of the corresponding eye in such a manner that a longitudinal direction of the second electrode elongates along the corresponding eye, each stimulator being configured to apply electrical stimulation to a human body by passing electricity between the first electrode and the second electrode, the second electrode including a conductive second electrode body and a conductive thin sheet accommodated along a longitudinal direction of the second electrode body.

(2) The cosmetic eye mask according to (1), wherein the thin sheet is a metal mesh.

(3) The cosmetic eye mask according to (1) or (2), wherein flexural rigidity of the second electrode body is greater than flexural rigidity of the thin sheet.

(4) The cosmetic eye mask according to any one of (1) to (3), wherein shore A hardness of the second electrode body is in a range from 30 Hs to 60 Hs.

(5) The cosmetic eye mask according to any one of (1) to (4), wherein the second electrode body is elastic and is curved along the longitudinal direction.

(6) The cosmetic eye mask according to any one of (1) to (5), wherein the second electrode body is mainly made of conductive medical-grade silicone.

(7) The cosmetic eye mask according to any one of (1) to (6), wherein the stimulator further includes: an operation unit; and a controller configured to switch mode to a first mode and to a second mode in response to an operation by a user on the operation unit, the first mode and the second mode differing in muscles around the eyes to which greater electrical stimulation is applied.

(8) The cosmetic eye mask according to (7), wherein the controller switches the mode to the first mode and to the second mode by switching an electrode to which a voltage is first applied between the first electrode and the second electrode.

(9) The cosmetic eye mask according to (7) or (8), wherein the controller is configured to apply greater electrical stimulation to temporalis muscle in the first mode and to orbicularis oculi muscle in the second mode.

(10) The cosmetic eye mask according to any one of (1) to (9), wherein the second electrode further includes a flexible substrate accommodated in the second electrode body, the flexible substrate being equipped with a film heater.

Of course, the present disclosure is not limited to these.

Finally, various embodiments of the present disclosure have been described, but these are presented as examples and are not intended to limit the scope of the disclosure. Novel embodiments can be implemented in various other forms, and various omissions, replacements, and modifications can be made within the scope of the spirit of the disclosure. The embodiments and its modifications are included in the scope and the spirit of the disclosure and are included in the scope of the invention described in claims and the equivalent scope thereof.

What is claimed is:

1. A cosmetic eye mask used in a manner such that the cosmetic eye mask is brought into contact with an area around eyes, the cosmetic eye mask comprising:
a mask body including accommodations, the accommodations being disposed at positions corresponding to positions around respective eyes; and
stimulators accommodated in the respective accommodations, each stimulator including:
a first electrode disposed at a position corresponding to a position on a lateral side of a corresponding eye and at an outer part of a face; and
a second electrode disposed at a position corresponding to a position on a lower side of each eye in such a manner that a longitudinal direction of the second electrode elongates along each eye, each stimulator being configured to apply electrical stimulation to a human body by passing electricity between the first electrode and the second electrode, the second electrode including a conductive second electrode body and a conductive thin sheet accommodated along a longitudinal direction of the second electrode body.

2. The cosmetic eye mask according to claim 1, wherein the thin sheet is a metal mesh.

3. The cosmetic eye mask according to claim 1, wherein flexural rigidity of the second electrode body is greater than flexural rigidity of the thin sheet.

4. The cosmetic eye mask according to claim 1, wherein shore A hardness of the second electrode body is in a range from 30 Hs to 60 Hs.

5. The cosmetic eye mask according to claim 1, wherein the second electrode body is elastic and is curved along the longitudinal direction.

6. The cosmetic eye mask according to claim 1, wherein the second electrode body is mainly made of conductive medical-grade silicone.

7. The cosmetic eye mask according to claim 1, wherein the stimulator further includes:
an operation unit; and
a controller configured to switch mode to a first mode and to a second mode in response to an operation by a user on the operation unit, the first mode and the second mode differing in muscles around the eyes to which greater electrical stimulation is applied.

8. The cosmetic eye mask according to claim 7, wherein the controller switches the mode to the first mode and to the second mode by switching an electrode to which a voltage is first applied between the first electrode and the second electrode.

9. The cosmetic eye mask according to claim 7, wherein the controller is configured to apply greater electrical stimulation to temporalis muscle in the first mode and to orbicularis oculi muscle in the second mode.

10. The cosmetic eye mask according to claim 1, wherein the second electrode further includes a flexible substrate accommodated in the second electrode body, the flexible substrate being equipped with a film heater.
